(19) United States
(12) Patent Application Publication
(10) Pub. No.: US 2026/0085675 A1
(43) Pub. Date: Mar. 26, 2026

(54) Pumping Device

(71) Applicant: MEDELA HOLDING AG, Baar (CH)

(72) Inventors: Bryan D. Simmons, North Aurora, IL (US); Natalie J. Stevens, Spring Grove, IL (US); Daniel McGowan, Wauconda, IL (US); Shane P. Einloth, Chicago, IL (US); Daniel J. Wright, Carpentersville, IL (US)

(21) Appl. No.: 18/895,694

(22) Filed: Sep. 25, 2024

(51) Int. Cl. F04B 43/02; A61M 5/142; F04B 13/00; F04B 51/00

(52) U.S. Cl. CPC F04B 43/028; F04B 13/00; F04B 51/00; A61M 5/14224

(57) ABSTRACT

The invention relates to a pumping device for feeding liquid feed to a patient, which can be used and assembled economically. The pumping device has a pumping cartridge and a drive unit. The pumping cartridge provides a pumping chamber and a pumping channel and valve inlet and outlet locations. The drive unit comprises actuators for pumping and opening and closing the valves at the valve inlet and outlet locations and can releasably be connected with the pumping cartridge. While the pumping cartridge is a disposable part, the driving unit is provided for multiple uses for supplying batch of liquid feed to the patient.

The invention relates to a pumping device, in particular a pumping device having a pumping cartridge intended for pumping fluid, in particular enteral nutritional fluids for a premature patient as an example for the feed to be pumped by the pumping device.

### BACKGROUND OF THE INVENTION

The present invention refers to Neonatal Enteral Feeding for NICU (Neonatal Intensive Care Unit) applications. Patients (newborn infants) born prematurely and under other circumstances have not developed the ability to feed orally, because this requires a suck-swallow-breathe-mechanism that is developed very late in the womb. Therefore, premature patients have to be supported for feeding until they are able to feed autonomously.

Premature patients need to be fed with enteral nutritional feed, such as mom milk based or milk containing mixture. The milk has to be collected, stored and maintained in a safe way and periodically (e.g. every 3 hours) fed to the patient. This is done in NICU hospital wards.

In particular, milk is collected by the mom (e.g. at home) and is brought to the hospital (NICU hospital ward). At the hospital, the milk is stored and when required, a feed in prepared with the milk for being fed to the patients. Preparation of the feed may include mixing different milk collected at different times from one and the same mom or mixing milk collected from different moms and in addition usually preparation also includes the addition of fortification, i.e. addition of nutrients, for the patients to receive the required nutrient substances also in small volume of feed (because the patient is premature!). The feed is fed to the patient via a syringe/pump system.

The syringe is filled with the feed, pre-warmed to body temperature and is then connected to an enteral feeding pump. This pump drives the syringe to deliver the feed to the patient at a controlled rate.

WO 2016 14 96 97 A1 discloses a disposable metering chamber for medical applications, for example drug delivery. The prior art discloses a reciprocating pump apparatus including
a metering chamber capable of separating from the pump body for replacing the fluid line. A flexible diaphragm in the removable metering chamber body is in contact with a flexible diaphragm in the reusable pump body such that when the flexible diaphragm in the pump body expands and contracts due to an electrochemical reaction, so does the diaphragm in the metering chamber body. This expansion and contraction of the metering chamber diaphragm allows a fluid in the metering chamber to flow from the reservoir to the target. The reusable pump body comprises an ionic fluid in two half-cells, which are separated from each other by a selective ionic membrane. Application of current between electrodes in each of the two half-cells causes an ionic imbalance to form between the two half-cells resulting in ions and associated solvent molecules to flow across the membrane.

U.S. Pat. No. 8,974,416 B2 discloses a disposable cartridge intended for a metering pump for infusion consisting of just two components, namely, a hollow first component forming a pump body, and a second component collaborating with the hollow first component to form a pumping zone and comprising a flexible part. The second component further comprises a rigid part secured to the flexible part and able to be assembled with the hollow first component. The flexible part provides inlet and outlet valves which open and close due to a pressure difference.

### PROBLEM TO BE SOLVED

The pumps used for NICU applications are not specifically developed for feed, but are of the type developed to deliver medicine into the bloodstream (drug pumps). The syringe/pump system is very expensive, in view of its main scope of use (drug delivery). While this syringe/pump system provides a very high degree of precision, such a precision is not needed for the neonatal enteral feeding application. Use of drug pumps as usually done therefore causes a waste or not optimal use of hospital resources. In addition, the small volume afforded by most syringes requires the health care provider to make frequent changes of the syringe, as the largest syringe won't accommodate the entire patient populations' needs. This results in additional labeling and tracking of feed containers, waste of disposables, increased risk of misadministration, risk of contamination, and wasted labor in managing unneeded syringes. Setting up a syringe pump requires significant skill and manual dexterity to prevent use error. The syringe/pump system cannot draw a vacuum on their volume, this does not allow a complete feeding of all feed contained in the feed tubing set and results in feed waste. As the pump also has a mechanism that compresses the syringe, over use of syringes, caused by frequent changes of syringe in the pumps, could also cause faults that result in a run-away drive condition. Syringe pumps displacement vs. delivery volume ratio changes with the size of syringe. The feed rate is dependent on the pump knowing the syringe cross-sectional area. Therefore the pump has to be programmed with the correct syringe size before use and in case different syringes are available in the NICU ward, risk of mistakes (e.g. use of the wrong syringe, wrong programming, etc.) exists.

It is an object of the present invention to provide a pumping device with a disposable cartridge for medical use, which is simple, reliable, can be made of a minimum amount of component parts which, are easy to manufacture and assemble. The pumping device shall in particular provide an alternative solution to the known syringe-pump-system previously described and shall be useful for feeding patients with liquid feed.

### SUMMARY OF THE INVENTION

According to the invention, the aforementioned object is achieved by means of a pumping device comprising a pumping cartridge and a drive unit. According to a second aspect of the present invention, the same provides a pumping cartridge, which can form part of such pumping device.

According to the first aspect of the present invention there is provided a pumping device for pumping feed, comprising a pumping cartridge having a pumping chamber and an inlet port and an outlet port. Each of said inlet port and outlet port being in fluid communication with said pumping chamber. The pumping device furthermore has a drive unit adapted to be releasably coupled to the pumping cartridge, wherein the drive unit comprises an inlet valve actuator for opening or closing the inlet port, an outlet valve actuator for opening or closing the outlet port, and a pumping actuator for pumping a fluid away from the pumping chamber via the outlet port.

The pumping cartridge may have a cartridge housing. The cartridge housing may provide a cartridges interface and the pumping chamber. The cartridge housing can provide the inlet port and the outlet port, each of the ports may be in fluid communication with said pumping chamber via a pumping channel. This pumping channel is provided within the cartridge housing. The pumping chamber preferably is provided with volume varying means for varying the volume of the pumping chamber. Such means can be a piston and/or a membrane, which is adapted to at least in part move into the pumping chamber and being retracted therefrom. Naturally, the volume varying means are usually moved in a predetermined reciprocating manner in order to effect pumping of the liquid.

For directing the flow within the pumping cartridge, preferably an inlet valve location and an outlet valve location are defined within the cartridge housing. The inlet valve location is usually arranged in vicinity to the inlet port and upstream of the pumping chamber. The outlet valve location is provided downstream of the pumping chamber and preferably in vicinity of the outlet port. Those valve locations are provided for closing the pumping channel upstream or downstream of the pumping chamber. The valve location per se may not need to be provided to actually close the pumping chamber. Instead and according to the invention, they are preferably provided for closing and opening the pumping chamber and bring the pumping chamber in communication with the inlet port or outlet port, in combination with the valve actuators provided by a drive unit. Those valve actuators can be passive actuators which may e.g. open and close the inlet and outlet valve location in response to a pressure difference. The drive unit is adapted to be releasably coupled to said pumping cartridge. While the pumping cartridge per se may be the disposable part and may be disposed after use, the drive unit is usually provided to be reusable and can releasably be connected in a sequence with multiple disposable pumping cartridges.

The drive unit may have a drive interface adapted to abut against the cartridge interface. The drive unit has an inlet valve actuator which may be assigned to cooperate with the inlet valve location and e.g. adapted to move beyond the drive interface and into the cartridge housing for providing an inlet valve. Said inlet valve is usually capable of fully opening and closing the pumping channel upstream of the pumping chamber. Moreover, the drive unit has an outlet valve actuator which may be assigned to cooperate with the outlet valve location within the cartridge housing and adapted to move beyond the drive interface and into the cartridge housing for providing an outlet valve. Said outlet valve may be capable of fully closing the pumping channel downstream of the pumping chamber.

The inlet and the outlet actuators are preferably adapted to advance the membrane into the pumping cartridge to make contact with an opening or mouth of the inlet port or the outlet port, respectively.

The drive unit furthermore has pumping actuator, in particular in form of a reciprocating piston assigned to cooperate with the pumping chamber and e.g. adapted to move beyond the drive interface and into the housing for varying the volume of the pumping chamber. The drive unit may have at least one drive means like a motor, in particular an electric motor for actuating at least one of the inlet valve actuator, the outlet valve actuator and the pumping actuator.

For releasably coupling the drive unit to the pumping cartridge, snapping means may be provided. Those snapping means likewise assist proper alignment of the pumping cartridge relative to the drive unit, such that the above-mentioned actuators of the drive unit are properly positioned relative to each of the pumping chamber, the inlet valve location and the outlet valve location, respectively.

According to a preferred embodiment, the membrane covers the pumping chamber and cooperates with the pumping actuator for pumping the feed. Further, the membrane may cover the inlet valve actuator opening and the outlet valve actuator opening.

According to a preferred embodiment, the cartridge housing and the drive unit housing are connected with each other, with the membrane sandwiched therebetween.

According to a preferred embodiment the cartridge has a first housing element and a second housing element, which housing elements are connected with each other, with the membrane sandwiched therebetween. The first housing element according to this preferred embodiment provides the cartridge interface, which cartridge interface is provided with a drive opening adapted to receive the pumping actuator, an inlet valve actuator opening adapted to receive the inlet valve actuator and an outlet valve actuator opening adapted to receive the outlet valve actuator.

The aforesaid membrane is preferably exposed at the cartridge interface in the drive opening, the inlet valve actuator opening and the outlet valve actuator opening. The membrane is furthermore arranged to cover the pumping chamber and at least a part of the pumping channel. Usually, the membrane is sandwiched between the cartridge housing and the drive unit housing to cover the entire path of the liquid to be conveyed through the pumping cartridge in a plane lying parallel to sandwiching surfaces of the cartridge housing and the drive unit housing. Preferably, at least the cartridge housing is a plate element, which has a rather simple configuration and may be provided with the openings mentioned above.

Preferably, the cartridge housing and the drive unit housing each provide a flat sandwiching surface abutting against each other with the membrane interdisposed therebetween. Preferably, the cartridge housing and the drive unit housing are each made of a plastic material.

Preferably, the membrane is sandwiched between the first housing element and the second housing element to cover the entire path of the liquid to be conveyed through the pumping cartridge in a plane lying parallel to sandwiching surfaces of the first and the second housing element. Preferably, at least the first housing element is a plate element, which has a rather simple configuration and may be provided with the openings mentioned above.

Preferably, the first housing element and the second housing element each provide a flat sandwiching surface abutting against each other with the membrane interdisposed therebetween.

Preferably, the first housing element and the second housing element are each made of a plastic material.

The pumping chamber, the pumping channel and the inlet and outlet ports are preferably provided by contours of the cartridge housing projecting a generally flat outer surface of the cartridge housing. This flat outer surface is arranged opposite to the flat sandwiching surface of said cartridge housing element. According to this preferred constitution, both, the cartridge housing and the drive unit housing can have rather slim constitution. Preferably, all projections providing the pumping chamber and the pumping channel are only provided on one, i.e. the outer surface of the generally flat cartridge housing. The drive unit housing can be completely flat, i.e. without any projections beyond the cartridge interface on one side and the flat sandwiching surface of said cartridge housing on the other side. The inner sandwiching surface may only be projected by fasteners provided on the outer circumference of the cartridge housing.

Further, the pumping chamber, the pumping channel and the inlet and outlet ports are preferably provided by contours of the second housing element projecting a generally flat outer surface of the second housing element. This flat outer surface may be arranged opposite to the flat sandwiching surface of said second housing element. The first housing element and the second housing element can have rather slim constitution. Preferably, all projections providing the pumping chamber and the pumping channel are only provided on one, i.e. the outer surface of the generally flat second housing element. The first housing element can be completely flat, i.e. without any projections beyond the cartridge interface on one side and the flat sandwiching surface of said first housing element on the other side. The inner sandwiching surface may only be projected by fasteners provided on the outer circumference of the first housing element.

The cartridge housing and the drive unit housing may be joined by snapping and/or gluing and/or welding. The membrane may just be sandwiched between the two housing elements thereby sealing off the pumping chamber and the pumping channel. Alternatively, the membrane may be glued or welded to be connected with one of the housings, preferably with the cartridge housing to thoroughly seal the pumping chamber and the pumping channel.

Further, the first housing element and the second housing element may be joined by snapping and/or gluing and/or welding. The membrane may just be sandwiched between the two housing elements thereby sealing off the pumping chamber and the pumping channel. Alternatively, the membrane may be glued or welded to be connected with one of the housing elements, preferably with the second housing element to thoroughly seal the pumping chamber and the pumping channel.

The above-described solution and the preferred embodiments thereof provide a pumping device which can be assembled easily and manufactured at low costs. The pumping cartridge can be fully made of plastic material. Both of the aforesaid housings may be injection molded. The membrane is the only part, which need to be connected between and/or with one of the housings and/or housing elements. Due to the membrane covering the pumping chamber and the pumping channel, the drive unit never gets into contact with the liquid to be pumped. The pumping device shall be used for metering the amount of liquid pumped. For this, the inlet and outlet valves may be closed and opened in a pre-determined sequence and synchronized in accordance with the movement of the pumping actuator/pumping piston.

As mentioned above, the inlet and outlet valve actuators may be passive actuators and may operate to open and close the respective inlet and outlet ports without being driven by a drive means and e.g. only by the pressure difference of the feed to be pumped within the pumping cartridge. In case only one drive means or motor is provided in the drive unit to also drive the inlet and outlet valve actuators, cam drives for each of the outlet valve actuator, the inlet valve actuator and the pumping actuator can be provided which will lead to a forced and synchronized movement of each of those actuators. In the course of such synchronized movement, the inlet valve will be closed when advancing the pumping actuator into the cartridge housing for expelling fluid out of the pumping chamber while the outlet valve is open whereas the outlet valve will be closed as the pumping actuator is retracted from the cartridge housing to allow the pumping volume to expand and allow fluid to enter through the inlet port as the inlet valve is opened by them. Each stroke of a cyclic pumping actuator can thereby be assigned to a specific volume of fluid pumped out of the pumping cartridge and through the outlet port. With this information, a controller of the drive unit may generate a signal indicative of the overall volume pumped and/or the actual volume flow.

Alternatively, the drive unit same can likewise be attained with two or more different drive means or motors. For example, a first motor can drive and thereby control the movement of the outlet and inlet valve actuators while another motor will provide reciprocating movement of the pumping actuator. Both, the outlet and inlet actuators as well as the pumping actuator are usually driven in a linear fashion for introducing the actuators into the cartridge housing and retracting the same therefrom.

The pumping device may have the following additional features and functionalities. According to a preferred aspect of the present invention, the pumping cartridge has a purging chamber arranged upstream of the pumping chamber and provided with a purged valve. This purge valve is adapted to be moved into a pumping position, in which the inlet port is in fluid communication with the pumping chamber. Furthermore, the purged valve can be brought into a purging position, in which an air inlet is in fluid communication with the pumping chamber. Such functionality allows to expel the entire liquid out of the pumping cartridge. In case the desired volume of liquid feed has been advanced towards the patient, the purge valve may be activated to shift the same from the pumping position into the purging position. As the pumping actuator is still active, no further liquid feed is introduced into the pumping cartridge through the inlet port. Instead, air is introduced into the pumping cartridge through the air inlet and advanced through the pumping channel. The air introduced will advance all liquid received within the cartridge housing towards the outlet port.

For automatically actuating the purge valve, the drive unit preferably has a purge valve drive adapted to move the purge valve between the pumping position and the purging position. The purge valve drive is usually contained in the drive unit and adapted to be non-rotatably connected with a valve actuator contained in the pumping cartridge when joining the drive unit and the pumping cartridge.

To avoid air from being delivered to the patient, an air detector is preferably arranged or arrangeable downstream of the pumping chamber. This air detector can be arranged in vicinity to the outlet port, i.e. downstream or upstream of the outlet port. The air detector may be mounted between the outlet port and a discharge tube, which is in fluid communication with the outlet port to transport the liquid feed to the patient, or at the end of such a discharge tube.

The drive unit preferably has a controller adapted to control the purge valve drive in response to a signal of said air detector indicative of the present of air detected by the air detector. Thus and in case purging leads to air exiting the pumping cartridge, such incidence will be detected with the consequence that purging of the cartridge is stopped before air is pumped into the patient to be fed.

According to another preferred aspect of the present invention, the drive unit has a controller with a priming memory, which is adapted to store a predetermined priming volume. This volume corresponds at least to the volume to be pumped for filling tubing downstream of the outlet end with liquid. The priming memory automatically assists proper priming of this tubing. For this, the controller is adapted to control a priming sequence in which the pumping device is activated to pump the predetermined priming volume in order to expel all or at least almost all air out of the fluid path downstream of the outlet port. The predetermined priming volume may as well contain at least a volume fraction corresponding to the volume of the pumping chamber and the pumping channel within the pumping cartridge.

For activating the priming sequence, a user may direct the controller to start this sequence e.g. via an interface provided by the drive unit or a remote control like a mobile phone or the like which may be connected to the drive unit wirelessly. The priming memory may contain various pre-stored priming volumes depending on the length and the inner diameter/lumen of the feeding tube. The priming memory allows for automatically filling the fluid path to the patient possibly including the fluid path within the pumping cartridge prior to the regular pumping routine for feeding the patient. In other words, the priming sequence can be used to prepare the pumping device for operation thereby securing an operation in which no or only a minimum amount of air is advanced into the patient. Different sets of tubing with respect to length and/or lumen may each be provided with an identification like a QR-code which can be automatically recognized by a the controller, in particular the hand held of the user and can be in data communication with the control of the drive unit.

Alternatively, the pumping device may comprise a priming sensor couplable to a tube connectable to said outlet port. The drive unit according to this preferred embodiment has a controller which is adapted to control a priming sequence. In said priming sequence, the controller orders the at least one motor to pump liquid and to stop pumping upon receipt of a signal of the priming sensor indicative of the presence of liquid. Naturally, respective priming sensor is usually coupled near or at the end of the discharge tube. Data connection between the control and the priming sensor may be provided by a wire or wirelessly.

According to a further preferred embodiment of the present invention, the pumping device furthermore has an occlusion sensor adapted to identify an occlusion downstream of the pumping chamber. Preferably, the drive unit has a controller which is adapted to output at least one signal indicating an occlusion. Thus, in case of an occlusion e.g. by a kinked discharge tube, the user of the pumping device is immediately notified by the signal that despite functionality of the pump no or an insufficient amount of liquid feed is advanced towards the patient. The occlusion sensor may be assigned to the discharge tube. Specifically, the aforementioned discharge tube may be introduced into or through the occlusion sensor, which sensor cooperates with the elastic lumen of the discharge tube. The occlusion sensor may be bias against the tube to provide a signal indicative of an internal pressure within the tube. In case of an occlusion downstream of the location of the occlusion sensor, the lumen will be expanded to a higher degree than if no occlusion would exist downstream of the occlusion sensor. This different inner pressure can be sensed by the occlusion sensor. The occlusion sensor may as well be provided within the pumping cartridge and provided downstream of the outlet port and upstream of the pumping chamber. Such occlusion sensor may likewise use the membrane to sense different pressure conditions within the pumping channel downstream of the pumping chamber in order to notify pressure conditions within the fluid path justifying a conclusion of an occlusion.

Apart from the detection of an occlusion, the occlusion sensor may contribute to assess the fluid volume flow through the pumping device. As discussed above, the occlusion sensor is in particular adapted to detect an internal lumen pressure within a flexible tube like e.g. the discharge tube. The occlusion sensor may likewise detect an internal pressure within the pumping channel. The occlusion sensor may be assigned to the pumping cartridge. Based on the pressure signal detected by the occlusion sensor, the same may identify a stroke of the volume varying means assigned to the pumping chamber. In case each stroke is assigned to a specific constant and known volume output (stroke volume), the fluid volume flow can be assessed by multiplying the number of strokes counted by the occlusion sensor with the stroke volume. In other words, the occlusion sensor may assist to meter the fluid pumped though the pumping device.

The present invention furthermore provides a pumping cartridge, which can be used as the disposable part of the pumping device discussed above.

### BRIEF DESCRIPTION OF THE DRAWINGS

The present invention will be better understood with reference to the detailed description taken in combination with the drawings in which

FIG. 1 is a schematic view of a feeding system;

FIG. 2 is a perspective top view of the pumping device of the feeding system of FIG. 1;

FIG. 3 is a perspective top view of the pumping device of FIG. 2 from the other side;

FIG. 4 is a first sectional view along line IV-IV according to FIG. 2;

FIG. 5 is a sectional view along lines V-V example finding in particular a purge valve;

FIG. 6 is a top view of the purge valve according to FIG. 5 in a pumping position;

FIG. 7 is a perspective view in accordance with FIG. 6 for the purge valve in a purging position;

FIG. 8 is a cross-sectional view along line VIII-VIII in FIG. 2 exemplifying an occlusion sensor;

FIG. 9a, b show a schematic view of an alternate occlusion sensor;

FIG. 10 shows a schematic cross sectional view of the drive unit of the pumping device of FIGS. 2, 3;

FIG. 11 shows a top view of a drive interface of the drive unit of FIG. 10;

FIG. 12 shows a perspective partially exploded view of the actuators of the drive unit of FIGS. 10, 11 and the motors provided for operating those actuators;

FIG. 13 schematically exemplifies the controller of the feeding system of FIG. 1;

FIG. 14 a graph on the pressure signal of the occlusion sensor.

### DETAILED DESCRIPTION OF THE PREFERRED EMBODIMENTS

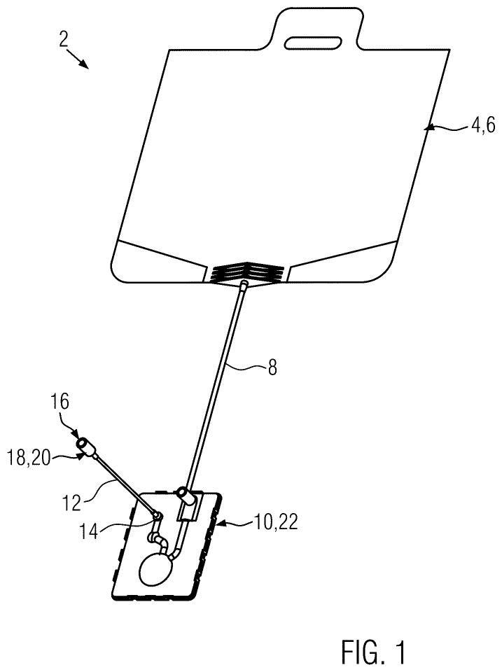

Referring to the drawings, there is shown in FIG. 1, a schematic of the feeding system of the present invention generally identified with reference number 2 and comprising a reservoir 4 in form of a flexible bag 6, the outlet side thereof being connected to a supply tube 8 which provides a fluid connection between the reservoir 4 and a pumping device 10. Downstream of said pumping device there is provided a discharge tube 12, which has an inlet end 14 and an outlet end 16.

In the shown embodiment, the reservoir 4 contains liquid feed for feeding a patient. Accordingly, the inlet end 14 receives said liquid feed from the reservoir 4 whereas the outlet end of the discharged tube 12 is adapted to discharge the liquid feed, preferably by introducing the discharge tube 12 and thus the liquid feed directly into the patient.

Reference number 18 identifies a purge sensor assigned to the outlet end of the discharge tube 12. Respective purge sensor 18 is provided at or in close vicinity to the outlet end 16. Reference number 20 identifies a priming sensor at or in close vicinity to the outlet end 16. The purge sensor 18 and the priming sensor 20 may be embodied in a single sensor arrangement.

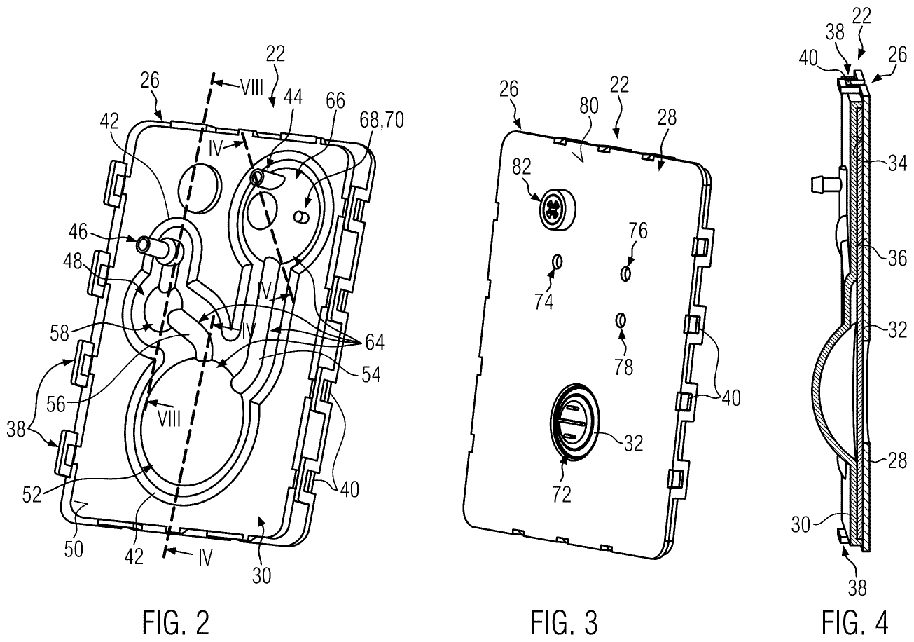

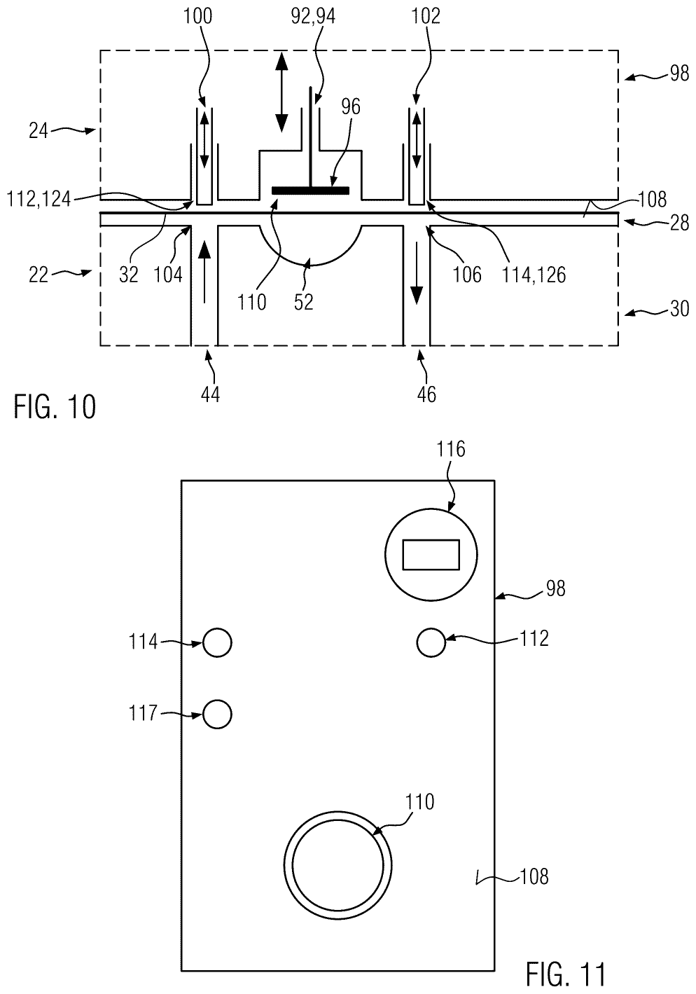

FIGS. 2 to 4 exemplify details of a pumping cartridge 22 of the pumping device 10 of FIG. 1. As schematically shown in FIG. 10 respective pumping cartridge 22 is adapted to be releasably connected to a drive unit 24. In other words, the pumping cartridge 22 provides the disposable part of the pumping device 10, whereas the drive unit 24 is intended for being reused plural times. The feeding system 2 may have a reservoir 4 like e.g. a bag of liquid feed to be fed to the patient. The pumping cartridge 22 may be connected via the tube 8 to the reservoir, so that after use reservoir, tube 8 and pumping cartridge 22 are dispensed with, while the drive unit 24 is consecutively releasably connected to multiple of the pumping cartridges.

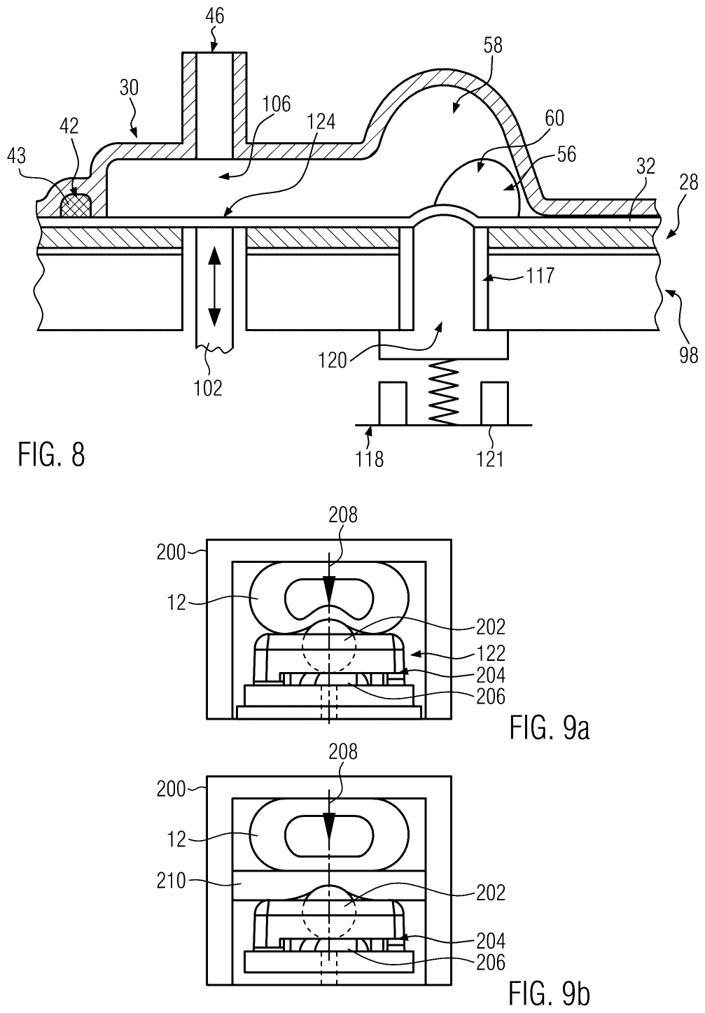

The pumping cartridge 22 has a cartridge housing 26 being composed of total three components i.e. a first housing element 28, a second housing element 30 and a membrane 32, which membrane 32 is sandwiched between the first and the second housing elements 28, 30, respectively. As shown in FIG. 4, the membrane 32 abuts a first sandwiching surface of the first housing element 28 and a second sandwiching surface 36 of the second housing element 30. As evident from FIG. 4, those inner sandwiching surfaces 34, 36 are each completely flat. Around the outer circumference of at least one of the first and the second housing elements 28, 30 there are provided snapping elements 4 connecting the two housing elements 28, 30 with each other with the membrane 32 sealed between those two housing elements 28, 30 respectively. In the specific embodiment, the first housing element 28 has U-shaped receptacles 38 receiving and encompassing bosses 40 at the outer circumference of the second housing element 30. The membrane 32 may be sealed between the two inner sandwiching surfaces 34, 36 by connection of the two housing elements 28, 30 respectively and this just be sandwiching. In the present embodiment, the second housing element 30 is provided with a sealing grove 42, which contains sealant 43 (see FIG. 8) for sealing the membrane 32 against the second housing element.

As evident from FIG. 2, an inlet port 44 and an outlet port 46 are provided within a sealed membrane area 48 surrounded by the sealing grove 42. The inlet port 44 and an outlet port 46 project perpendicular from a generally flat outer surface 50 of the second housing element 30. Reference number 52 identifies a pumping chamber connected to the inlet port 44 by an upstream pumping channel section 54 and connected to the outlet port 46 by a downstream pumping channel section 56, which is interrupted by a occlusion sensor chamber 58 providing an occlusion sensor location 60.

The upstream and downstream pumping channel sections 54; 56 define a pumping channel 62. The pumping channel 62, the occlusion sensor chamber 58 and the pumping chamber 52 are each provided by contours projecting the flat outer surface 50 of the second housing element 30.

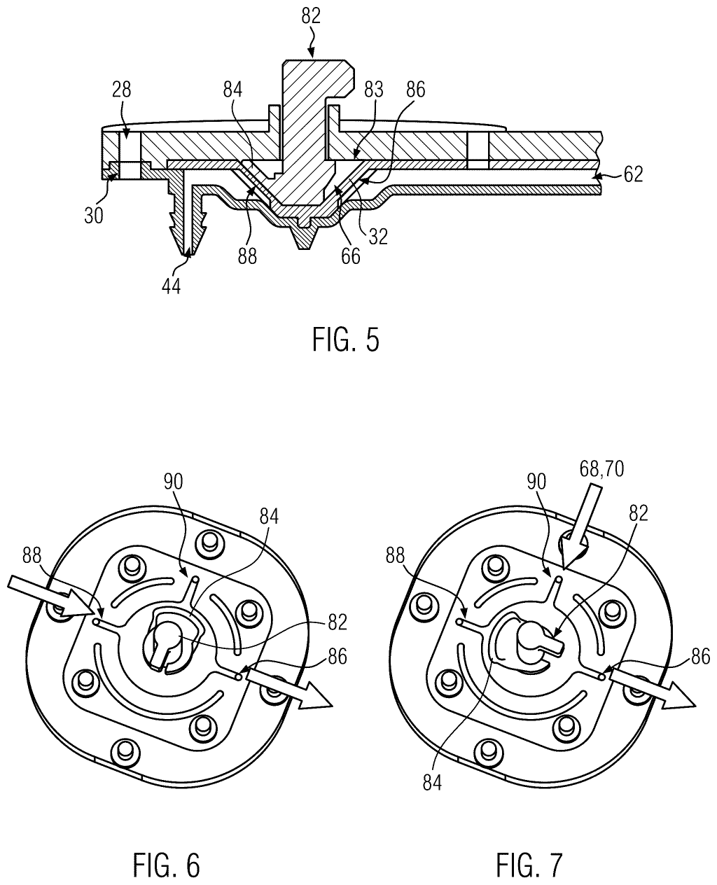

The inlet port 44 projects from a purge chamber 66, the functionality thereof, being further described by referring to FIGS. 5-7. On the outer circumference of said purge chamber 66 there is provided a purge inlet 68 in form of an air inlet 70.

As shown in FIG. 3, the first sandwiching surface has a pumping actuator opening 72, an inlet valve actuator opening 74, an outlet valve actuator opening 76 and an occlusion sensor opening 78. Those openings 72-78 are covered by the membrane 32. As the membrane 32 is provided between the two housing elements 28, 30 and sealingly secured to the second housing element 30, the membrane 32 covers the pumping channel 62, the pumping chamber 52 and the occlusion sensor chamber 58; see FIGS. 4 and 8. As further evident from FIG. 5, the membrane 32 is likewise projected into the frustoconical purge chamber 66.

FIG. 3 shows the first housing element 28 with its flat cartridge interface 80, in which the above-mentioned openings 72-78 are recessed and sealed on the inner side by the membrane 32. The cartridge interface 80 is projected by a purge valve actuator 82, which will be described hereinafter in particular by referring to FIGS. 5-7.

As evident from FIG. 5, the purge valve actuator 82 has a valve arm 84 formed in an oblique way in accordance with the inner circumferential oblique surface of the frustoconical purge chamber 66. The purge chamber 66 has a purge chamber outlet opening 86, a purge chamber feed inlet opening 88, both being shown in FIG. 5 and a purge chamber purge inlet opening 90, which is essentially provided in accordance with the purge chamber feed inlet opening 88 but arranged at a 90° offset in clockwise direction as seen in FIGS. 6 and 7, respectively.

In FIG. 5, the valve arm 84 presses the membrane 32 against the purge chamber feed inlet opening 88 thereby closing this opening 88. Due to the flexibility of the membrane 32 following this movement imposed by the valve arm 84, the purge chamber purge inlet opening 90 is open in the position of the purge valve actuator 82 as shown in FIGS. 5 and 7. This position is a purge position, in which the purge inlet 68 is via the purge chamber 66 in fluid communication with the purge chamber outlet opening 86 and thus with the pumping channel 62 and thus the pumping chamber 52. FIG. 6 shows the pumping position of the purge valve actuator 82, in which the purge chamber feed inlet opening 88 is open and the purge chamber purge inlet opening 90 is closed by the valve arm 84 in combination with the membrane 32. Thus, the inlet port 44 is in fluid communication with the pumping chamber 52.

FIG. 10 is a schematic view showing the pumping cartridge 22 and the drive unit 24 in a simplified sectional view. In the middle of FIG. 10, the pumping chamber 52 can be seen to be covered by the membrane 32. Opposite to the pumping chamber 52 there is provided a pumping actuator 92 in form of a piston 94 which moves in a reciprocating fashion and thus along with the membrane 32 provides volume varying means 94 for varying the effective volume within the pumping chamber 52. The reciprocating movement of the piston 94 is shown to be guided by a drive unit housing 98, which likewise guides a reciprocating movement of an inlet valve actuator 100 and an outlet valve actuator 102. Opposite to the inlet valve actuator 100 the cartridge housing, specifically the second housing part 30 provides an inlet valve location 104. Reference number 106 identifies an outlet valve location provided by the second housing part 30.

As can be seen from FIGS. 10 and 11 in combination, the drive unit housing 98 provides a drive interface 108 having plural openings, i.e. a drive side pumping actuator opening 110 receiving and guiding the pumping actuator 92, a drive side inlet valve actuator opening 112 and a drive side outlet valve actuator opening 114, each receiving and guiding one of the inlet valve actuator 100 and outlet valve actuator 102, respectively. Reference number 116 in FIG. 8 identifies a drive side occlusion sensor opening, which receives and guides an occlusion sensor identified in total with reference number 118 in FIG. 8. This occlusion sensor 118 contains a sensor contact element 120 biased towards the membrane 32 and cooperating with a sensor die 121. Due to the pretension of the sensor contact element 120 towards the occlusion sensor chamber 58, the membrane 32 bulges inwardly into said occlusion sensor chamber 58.

As already mentioned above, the valve actuators 100, 102 are adapted to move beyond the drive interface 108 and into the cartridge housing 26.

Preferably and exemplified in FIG. 8, the inlet and/or outlet valve location is provided by the inlet and outlet port 44, 46, respectively. In such an embodiment, the forward free end of the inlet valve actuator 100 cooperates with the opening of the inlet port 44 and the outlet valve actuator 102 cooperates with the opening of the outlet port 46 for opening and closing an inlet valve 122 or an outlet valve identified in FIG. 8 with reference number 124.

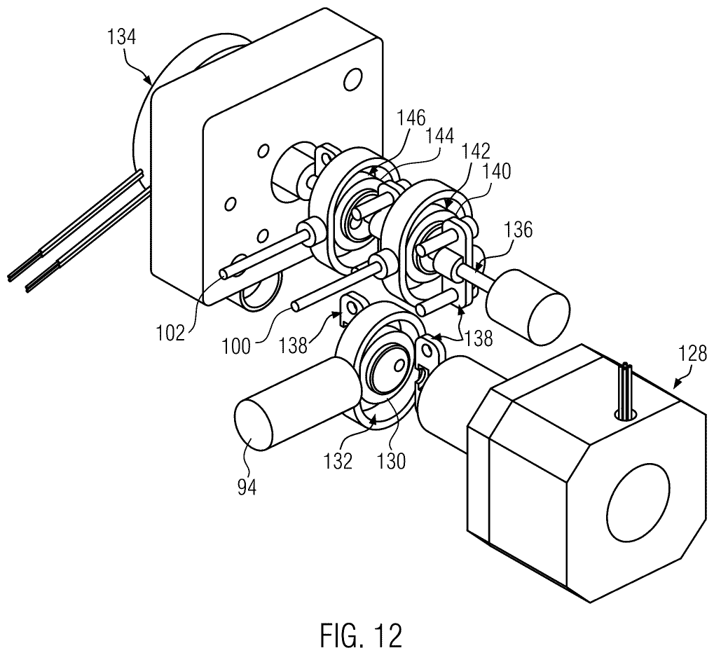

FIG. 12 is a schematic of the drive system of the drive unit 24. In this specific embodiment, a first motor 126 is provided rotatably driving a piston cam 128, which piston cam 128 is received within a piston cam receptacle 130 connected to the piston 94.

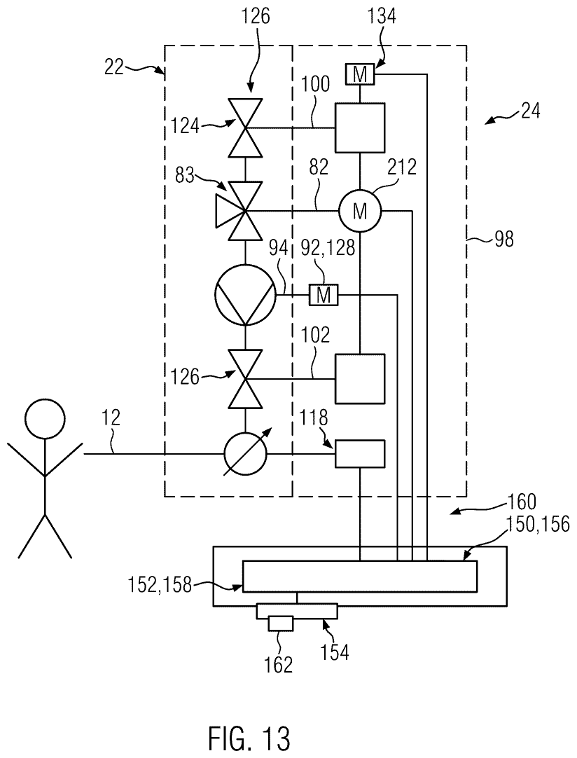

A second motor 132 rotatably drives a shaft 134 supported by bearings 136, which shaft 134 drives an inlet valve cam 138 received within an inlet valve cam receptacle 140 connected to the inlet valve actuator 100. In a respective fashion, an outlet valve cam 142 connected to the shaft 134 is received within an outlet valve cam receptacle 144, which is connected to the outlet valve actuator 102. The motors 126, 132 are synchronized by a controller identified with reference number 150 in FIG. 13 to provide a specific operation in which the inlet valve 122 is opened to permit liquid feed to flow into the pumping chamber 52. After that the inlet valve 122 is shut off or closed by advancing the inlet valve actuator 100 towards a mouth of the inlet port 44 which by interdisposing the flexible material of the membrane 32 is completely sealed by the membrane 32. Then the outlet valve 124 is opened while compressing the pumping chamber 52 by means of the piston 95 and the membrane 32.

Dislocation of the sensor contact element 120 of FIG. 8 towards the drive unit housing 98 is sensed by the occlusion sensor 118 and processed in the controller 150 as an indication of occlusion downstream of the occlusion sensor 118

While FIG. 12 shows an embodiment with two motors, it is evident that a single motor can drive a sample shaft 134 being connected to the three cams 128, 138, 142 moving the piston 94 and the inlet and outlet valve actuators 100, 102, respectively.

While the above description relates to an occlusion sensor 118 provided by the pumping cartridge 22, such occlusion sensor may likewise be provided separately and cooperate with the discharged tube 12 as exemplified in FIGS. 9a, 9b. FIG. 9a shows an occlusion sensor in which the sensors switch 122 is surrounded by a U-shaped sensor clamp 200, which forces the discharged tube 12 against the sensor switch 122. In this embodiment, the sensor switch 122 comprises a sphere 202 contained in a sensor housing 204, housing a sensor die 206. With increasing internal pressure within the lumen of the discharge tube 12, the sphere 202 will be forced in the direction of arrow 208 thereby forcing the sphere 202 against the sensor die 206, which will eventually will switch the sensor switch 122 thereby signaling the existence of an occlusion. The occlusion occurs if the pressure generated in the pumping chamber is not released by flow of the feed to be pumped.

FIG. 9b shows a variant of the embodiment of FIG. 9a in which an additional protective cover 210 is interdisposed between the tube 12 and the sphere 202.

As already mentioned above, FIG. 13 shows a controller 150 comprising a memory 152 and a controller interface 154, which controller interface 154 may be a user interface and/or a wirelessly interface for use in combination with e.g. a hand held control unit such as a mobile phone. The controller 150 may be contained in the pump unit 24 or may be contained in a separate controller housing connected with said pump unit 24 by wires 160 or wirelessly.

While the basic concept of pumping with the described embodiment has been explained above, the embodiment of the present invention provides further operational modes, which will be described hereinafter.

The embodiment is able to purge the discharged tube 12 and the volume of feed contained in the pumping cartridge as the operation of the feeding system 2 is terminated. Such purging will reduce the amount of liquid feed, which will have to be discharged in the discharge tube and in the pumping cartridge, which are both disposable and thus would not be used for feeding the patient.

At the end of feeding a batch of liquid feed with the feeding system 2, the purge valve 83 is activated by the purge valve actuator 82 which is driven by a separate purge valve motor 212 provided within the drive unit 24 by means of the controller 150—see FIG. 13. The controller continues to drive the piston 94 and the inlet and outlet valve actuators 100, 102. In other words, volumetric pumping yield is maintained. Each volume per stroke of the piston 94 is stored in the memory 152 of the controller. As the valve arm 84 is provided in the purging position (compare FIG. 7), air is introduced into the pumping channel 62, which air drives the liquid feed contained downstream of the purge inlet 68. As the pump is still activated, the remaining volume of liquid feed is advanced towards the outlet end 16 of the discharged tube 12. Here, the purge sensor 18 will notify arrival of the air in the lumen of the discharge tube 12 and will give a signal indicative of the presence of air to the controller 150. In view of this signal, the controller 150 will stop the pumping activity of the pumping cartridge 22.

Feeding of patient may require different sets of tubing with different tubing length and/or different lumen. Depending on the pumping yield, a different pumping cartridge may be connected to the drive unit 24. In other words, the user may select specific components to assemble the feeding system 2, wherein each of the respective components may have a different specific volume accounting to the flow path of the liquid feed from the reservoir 4 to the outlet end 16. Respective volume may be entered manually by transferring respective information from the package of each of the component. Alternatively, the packaging may contain a barcode, which can be used to read respective information electronically into the memory 152. With this information, the memory 152 can calculate or at least assess the overall volume of the flow path downstream of the purge inlet 68. Knowing the volumetric yield of each stroke of the piston 94, the controller 150 can calculate the number of strokes to purge or almost fully purge remaining liquid feed contained in the fluid path downstream of the purge valve 83.

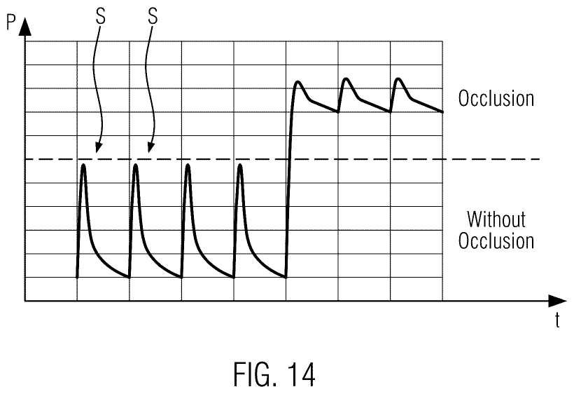

The number of strokes may be counted by the controller 150. Alternatively or additionally, the signal of the occlusion sensor 118 may be used to count the number of strokes in the controller 150. As evident from FIG. 14, each stroke S of the piston 94 will result in a specific pressure signal obtained by the occlusion sensor 18, which signal can be used to count the strokes.

The last three strokes in FIG. 14 correspond to a signal of the occlusion sensor 118 obtained at the incident of an occlusion downstream of the pumping chamber 52. A high pressure builds up which is noted by the occlusion sensor 118 and which is reported to the controller 150 to release a signal indicative of an occlusion. For example, the controller 150 may release an acoustic and/or optical warning notifying the user of the feeding system 2 that liquid feed is not advanced towards the patient as intended.

The above described memory 152 containing and/or receiving information on the specific volume of a specific component may likewise be used as a priming memory 158 for a priming sequence. Such a priming sequence may be initiated by the user via the user interface, which could e.g. comprise a priming button 162. The priming volume stored within the priming memory 158 corresponds to the volume of the feeding system 2, which shall be filled at least downstream of the outlet opening 46 of the pumping cartridge 22 and may as already described for the purge volume, in addition to the feed volume, which can be contained in the pumping cartridge 22 in particular in case multiple cartridge components may be used for assembling the feeding system 2. Knowing the overall volume, which needs to be primed, the controller can activate the pump in the priming sequence and control the priming sequence such that the pumping device 10 is activated to pump the predetermined priming volume of the liquid.

The priming sequence will fill the tubing with liquid feed before actually starting feeding of the patient. The priming sequence reduces the amount of air delivered to the patient when the pumping device 10 is activated at the beginning of supplying a batch of liquid to the patient. Thanks to the priming sequence, feeding of a patient can be started if at least almost the entire feeding system up to the outlet end 16 has been filled with liquid feed.

In addition or alternatively to the calculation of the priming volume described above, the priming sensor 20 may be adapted to identify the presence of liquid which approaches the outlet end 16 and drives air out of the discharge tube 12. Thus, the priming sensor 20 may provide a signal indicative of the presence of the liquid at the outlet end 16 and thereby stop the priming sequence by stopping the pumping device 10.

The purge sensor 18 and/or the priming sensor 20 can be any sensor for example an optical, sonic or temperature sensor, which sensor is located near or at the outlet end 16 to send a signal to the pumping device 10 to automatically stop the priming sequence or the purging phase.

### REFERENCE SIGNS

2 feeding system
4 reservoir
6 bag
8 supply tube
10 pumping device
12 discharge tube
14 inlet end
16 outlet end
18 purge sensor
20 priming sensor
22 pumping cartridge
24 drive unit
26 cartridge housing
28 first housing element
30 second housing element
32 membrane
34 first sandwiching surface
36 second sandwiching surface
38 receptacle
40 boss
42 sealing groove
43 sealant
44 inlet port
46 outlet port
48 sealed membrane area
50 outer surface
52 pumping chamber
54 upstream pumping channel section
56 downstream pumping channel section
58 occlusion sensor chamber
60 occlusion sensor location
62 pumping channel
64 contours
66 purge chamber
68 purge inlet
70 air inlet
72 pumping actuator opening
74 inlet valve actuator opening
76 outlet valve actuator opening
78 occlusion sensor opening
80 cartridge interface
82 purge valve actuator
83 3-way-purge valve
84 valve arm
86 purge chamber outlet opening
88 purge chamber feed inlet opening
90 purge chamber purge inlet opening
92 pumping actuator
94 piston
96 volume varying means
98 drive unit housing
100 inlet valve actuator
102 outlet valve actuator
104 inlet valve location
106 outlet valve location
108 drive interface
110 drive side pumping actuator opening
112 drive side inlet valve actuator opening
114 drive side outlet valve actuator opening
116 drive side occlusion sensor opening
118 occlusion sensor
120 sensor contact element
121 sensor die
122 sensor switch
124 inlet valve
126 outlet valve
128 first motor
130 piston cam
132 piston cam receptacle
134 second motor
136 shaft
138 bearing
140 inlet valve cam
142 inlet valve cam receptacle
144 outlet valve cam
146 outlet valve cam receptacle
150 controller
152 memory
154 controller interface
156 volume flow assessment means
158 priming memory
160 wire
162 priming button
200 sensor clamp
202 sphere
204 sensor housing
206 sensor die
208 arrow
210 protective cover
212 purge valve motor

We claim:

1. A pumping device for pumping feed, comprising
a pumping cartridge having a pumping chamber and an inlet port and an outlet port, each of said inlet port and outlet port being in fluid communication with said pumping chamber,
a drive unit adapted to be releasably coupled to the pumping cartridge, wherein the drive unit comprises
an inlet valve actuator for opening or closing the inlet port,
an outlet valve actuator for opening or closing the outlet port,
and a pumping actuator for pumping a fluid away from the pumping chamber via the outlet port.

2. The pumping device according to claim 1, wherein the drive unit comprises at least one drive means, for actuating at least one of the inlet valve actuator, the outlet valve actuator and the pumping actuator.

3. The pumping device according to claim 1, wherein the pumping cartridge has a cartridge housing and a flexible membrane delimiting the pumping chamber, and wherein the drive unit has a drive unit housing, wherein the cartridge housing and drive unit housing are connected with each other with the membrane sandwiched there between.

4. The pumping device according to claim 3, wherein the pumping cartridge housing has a pumping actuator opening, which pumping actuator opening being adapted to receive the pumping actuator.

5. The pumping device according to claim 4, wherein the membrane covers the pumping chamber and cooperates with the pumping actuator for pumping the feed.

6. The pumping device according to claim 4, wherein the inlet port is assigned to an inlet valve actuator opening that cooperates with the inlet valve actuator for opening or closing the inlet port and the outlet port is assigned to an outlet valve actuator opening that cooperates with the outlet valve actuator for opening or closing the outlet port.

7. The pumping device according to claim 6, wherein the inlet valve actuator opening and the outlet valve actuator opening are covered by the membrane.

8. The pumping device according to claim 1, wherein said drive unit has a controller with a priming memory, which memory is adapted to store a predetermined priming volume corresponding to the volume to be pumped for filling tubing downstream of the outlet opening with liquid and that the controller is adapted to control a priming sequence, in which the pumping device is activated to pump the predetermined priming volume of liquid.

9. The pumping device according to claim 1, further comprising a priming sensor couplable to a tube connectable to said outlet port, wherein said drive unit has a controller adapted to control a priming sequence, in which the controller orders to pump liquid and to stop pumping upon receipt of a signal of the priming sensor indicative of the presence of liquid.

10. The pumping device according to claim 1, further comprising an occlusion sensor adapted to identify an occlusion downstream of the pumping chamber, wherein said drive unit has a controller adapted to output at least one signal indicating to the user an occlusion

11. The pumping device according to claim 1, further comprising fluid volume flow assessment means adapted to assess the fluid volume flow of the fluid pumped through the pumping device on the basis of a varying volume of the pumping chamber.

12. The pumping device according to claim 10, wherein the occlusion sensor is adapted to detect an internal pressure within a flexible tube connected to outlet port or within the pumping channel to identify a stroke of the volume varying means of the pumping chamber, and wherein the fluid volume flow is assessed on the basis of the varying volume of the pumping chamber per stroke and the number of strokes detected by the occlusion sensor.

13. A pumping cartridge adapted to be releasably coupled to a drive unit, the pumping cartridge having a pumping chamber and an inlet port and an outlet port, each of said intel port and outlet port being in fluid communication with said pumping chamber, the pumping cartridge further having a releasable connection for connecting the pumping cartridge to a drive unit.

14. The pumping cartridge of claim 13, wherein the inlet port is connected or is connectable to a supply tube that is in turn connected to a reservoir and the outlet port is connected or is connectable to a delivery tube for supplying feed to a patient.

15. The pumping cartridge of claim 13, further comprising a cartridge housing providing pumping channel connecting the inlet and outlet ports, wherein the pumping chamber, the pumping channel and the inlet and outlet ports are provided by contours of the cartridge housing projecting a generally flat outer surface of the cartridge housing.

16. A pumping cartridge of claim 13 comprising a cartridge housing, which cartridge housing provides the pumping chamber and the inlet port and the outlet port, each of said ports being in fluid communication with said pumping chamber via a pumping channel provided within said cartridge housing,
wherein the cartridge housing has a first housing element and a second housing element, which housing elements are connected with each other with a membrane sandwiched there between,
wherein said membrane provides volume varying means for varying the volume of the pumping chamber,
wherein the first housing element provides the cartridge interface, which cartridge interface is provided with an pumping actuator opening adapted to receive the pumping actuator,
wherein the second housing element provides said pumping chamber, said pumping channel and said inlet and an outlet ports.

17. The pumping cartridge according to claim 16, wherein the second housing element provides an inlet valve location and an outlet valve location for closing the pumping channel upstream or downstream of said pumping chamber,
wherein the cartridge interface of the first housing element is provided with an inlet valve actuator opening opposite to the inlet valve location and an outlet valve actuator opening opposite to the outlet valve location, and
wherein the inlet valve actuator opening and the outlet valve actuator opening are each covered by the membrane.

18. The pumping cartridge according to claim 16, wherein said cartridge interface of said first housing element is flat and wherein an inner sandwiching surface opposite to said cartridge interface is flat and abuts the membrane.

19. The pumping cartridge according to claim 16, wherein said second housing element has a generally flat inner sandwiching surface, which generally flat sandwiching surface abuts the membrane, and a outer surface opposite to said flat inner surface, which outer surface is projected by the inlet and outlet ports and is provided with contours providing said pumping chamber and said pumping channel.

20. The pumping cartridge according to claim 16, wherein said pumping cartridge has an purge inlet and a purge chamber arranged upstream of the pumping chamber and provided with a purge valve, which purge valve is adapted to be moved into a pumping position in which the inlet port is in fluid communication with the pumping chamber and a purging position in which said purge inlet is in fluid communication with the pumping chamber.

21. The pumping cartridge according to claim 16, wherein said second housing element has an occlusion sensor location in communication with said pumping channel and wherein said first housing element has an occlusion sensor opening, which occlusion sensor opening is covered by the membrane and arranged opposite to said occlusion sensor location.

22. A drive unit adapted to be releasably coupled to a pumping cartridge, wherein the drive unit comprises
an inlet valve actuator adapted to open or close an inlet port of the pumping cartridge when the drive unit is connected to the pumping cartridge,
an outlet valve actuator adapted to open or close an outlet port of the pumping cartridge when the drive unit is connected to the pumping cartridge,
and a pumping actuator adapted to pump a fluid away from a pumping chamber of the pumping cartridge via the outlet port of the pumping cartridge when the drive unit is connected to the pumping cartridge,
at least one drive means, adapted to actuate at least one of the inlet valve actuator, the outlet valve actuator and the pumping actuator
a releasable connection for connecting the drive unit to the pumping cartridge.

23. The pumping device of claim 1, wherein the pumping cartridge further comprises a cartridge housing, which cartridge housing provides a cartridge interface, the inlet port, the outlet port and a pumping channel providing the fluid communication between the inlet port and the outlet port,
wherein said pumping chamber is provided with volume varying means for varying the volume of the pumping chamber and wherein an inlet valve location and an outlet valve location are defined within the cartridge housing for closing the pumping channel upstream or downstream of the pumping chamber,
wherein the a drive unit further comprises a drive interface adapted to abut against the cartridge interface and at least one drive means for actuating at least one of the inlet valve actuator, the outlet valve actuator and the pumping actuator, and
wherein the inlet valve actuator is assigned to cooperate with the inlet valve location and adapted to move beyond the drive interface and into the cartridge housing for providing an inlet valve,
wherein the outlet valve actuator is assigned to cooperate with the outlet valve location and adapted to move beyond the drive interface and into the cartridge housing for providing an outlet valve,
and wherein the pumping actuator is assigned to cooperate with the volume varying means and adapted to move beyond the drive interface and into the cartridge housing for varying the volume of the pumping chamber.

24. The pumping device according to claim 23, wherein the cartridge has a first housing element and a second housing element, which housing elements are connected with each other with a membrane sandwiched there between, wherein the first housing element provides the cartridge interface, which cartridge interface is provided with an pumping actuator opening adapted to receive the pumping actuator, an inlet valve actuator opening adapted to receive the inlet valve actuator and an outlet valve actuator opening adapted to receive the outlet valve actuator, wherein the membrane is exposed at the cartridge interface in the pumping actuator opening, the inlet valve actuator opening and the outlet valve actuator opening and arranged to cover said pumping chamber and said pumping channel.

25. The pumping device according to claim 24, wherein said first housing element is a plate element.

26. The pumping device according to claim 23, wherein said first housing element and said second housing element each provide a flat sandwiching surfaces abutting against each other with the membrane sandwiched there between and wherein said pumping chamber, said pumping channel and said inlet and an outlet ports are provided by contours of said second housing element projecting a generally flat outer surface of said second housing element, which flat outer surface is arranged opposite to said flat sandwiching surfaces of said second housing element.
